(19) United States
(12) Patent Application Publication
(10) Pub. No.: US 2020/0054814 A1
(43) Pub. Date: Feb. 20, 2020

(54) INDIVIDUALIZED ULTRAFILTRATION PROFILES IN DIALYSIS

(71) Applicants: Rammah Abohtyra, Florence, MA (US); Yossi Chait, Longmeadow, MA (US); Christopher V. Hollot, East Hampton, MA (US); Joseph Horowitz, Amherst, MA (US); Michael J. Germain, Hampden, MA (US)

(72) Inventors: Rammah Abohtyra, Florence, MA (US); Yossi Chait, Longmeadow, MA (US); Christopher V. Hollot, East Hampton, MA (US); Joseph Horowitz, Amherst, MA (US); Michael J. Germain, Hampden, MA (US)

(21) Appl. No.: 16/545,184

(22) Filed: Aug. 20, 2019

Related U.S. Application Data
(60) Provisional application No. 62/720,012, filed on Aug. 20, 2018.

(51) Int. Cl. A61M 1/34; A61M 1/36; A61M 1/16

(52) U.S. Cl. CPC A61M 1/341; A61M 1/3609; G16H 10/60; A61M 2205/3344; A61M 1/1603; A61M 1/1647

(57) ABSTRACT

Hemodialysis is used to remove fluid from a patient suffering from end-stage renal kidney disease. An optimization-based approach including a design of an individualized ultrafiltration rate profile may be used to prevent hematocrit levels from exceeding a threshold, thereby preventing adverse effects in the patient. The individualized ultrafiltration rate profile may be based on previously obtained clinical data for the patient, a volume of fluid to be removed from the patient, a specified period of time for removal of the fluid, or a patient-specific fluid volume model. The ultrafiltration rate profile may be time-variant over the specified period of time.

### PRIORITY

This application claims the benefit of priority to U.S. Provisional Application No. 62/720,012, filed Aug. 20, 2018, titled “INDIVIDUALIZED ULTRAFILTRATION PROFILES IN DIALYSIS,” which is hereby incorporated herein by reference in its entirety.

### STATEMENT OF GOVERNMENT SUPPORT

This invention was made with government support under grant number K25 DK096006-01A1 awarded by the National Institutes of Health. The government has certain rights in the invention.

### BACKGROUND

According to some experts, there are hundreds of thousands of end-stage renal kidney disease (ESRD) patients requiring hemodialysis (HD). Despite significant advances in HD technology, only half of HD patients survive more than 3 years. Fluid management is one of the most challenging aspects of HD treatments, with serious implications for morbidity and mortality. Long- and short-term adverse outcomes in dialysis have been associated with intradialytic hypotension, a common dialysis complication and significant cause of morbidity. High ultrafiltration rates (UFRs) have been associated with intradialytic hypotension.

The current approaches for setting ultrafiltration rate (UFR) profiles in an HD machine use a few of fixed profiles. The profiles are not individualized to the patient and often are unsuccessful in keeping UFRs down. Some techniques use a feedback mechanism, but that can be risky and prone to failure.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a schematic diagram of a two-compartment fluid model describing fluid exchange between interstitial and intravascular compartments separated by a capillary membrane in accordance with some embodiments.

FIG. 2 illustrates an ultrafiltration rate profile and hematocrit informationover time in accordance with some embodiments.

FIG. 3 illustrates robustness hematocrit information for an ultrafiltration rate profile over time in accordance with some embodiments.

FIG. 4 illustrates boundary information for a state trajectory of a nonlinear model of an ultrafiltration rate in accordance with some embodiments.

FIG. 5 illustrates a flow chart showing a technique for removing fluid from a patient at a hemodialysis machine in accordance with some embodiments.

FIG. 6 illustrates a block diagram of an example of a machine upon which any one or more of the techniques discussed herein may perform in accordance with some embodiments.

### DETAILED DESCRIPTION

The systems and methods described herein optimize fluid management in hemodialysis according to a patient-specific (individualized) UFR profile. These systems and techniques may be used to minimize a maximal UFR needed to remove a prescribed volume of fluid within a set time subject to a time-varying critical hematocrit (Hct) constraint. The underlying fluid dynamics during HD may be described by a nonlinear fluid-volume model including intravascular and interstitial compartments. The nonlinear model may include parameters given in terms of nominal values with uncertainty ranges. The systems and methods described herein constrain states of the nonlinear model to within a specified physiologically plausible region, while meeting a critical Hct constraint. The systems and methods described herein may generate a constant UFR profile or a time-varying UFR profile.

The systems and methods described herein may include a real-time robust ultrafiltration (UFR) profile for a hemodialysis subjects based on measurements of hemoconcentration, identification of patient-specific fluid volume model during ultrafiltration, dialysis prescription, and patient-specific constraints. The nonlinear model may be approximated by a linear model.

An important measurement available to clinicians for guiding fluid removal during HD is blood pressure (BP), an informative characteristic of cardiovascular hemodynamics. However, current techniques include measuring BP every 20-30 minutes during HD, which cannot predict a future adverse event (e.g., intradialytic hypotension (IDH), a common complication of fluid removal) because BP is tightly regulated by several intrinsic control mechanisms that respond to the major perturbation of UF-induced blood volume reduction on time scales ranging from seconds to minutes. The systems and methods described herein provide results for removing fluid according to a personalized nonlinear model while reducing or avoiding adverse events.

Many end-stage kidney disease (ESKD) patients undergo HD treatments three times a week to remove excess fluid and waste products from the blood. In HD, fluid is removed from the intravascular space by ultrafiltration, and as a consequence, fluid from the interstitial moves into the intravascular space (vascular refilling), driven by hydrostatic and osmotic pressure gradients. Vascular refilling is critical for maintaining adequate blood pressure during dialysis. Whenever the rate of vascular refilling is smaller than the ultrafiltration rate, plasma volume declines, and hematocrit level rises.

In the systems and methods described herein, a robust, optimal ultrafiltration rate profile may be used to remove a specified amount of fluid for an individual patient whose physiological parameters are estimated online. The optimal profile is designed based on the estimated model, and robustness is achieved with respect to estimated parameter variability.

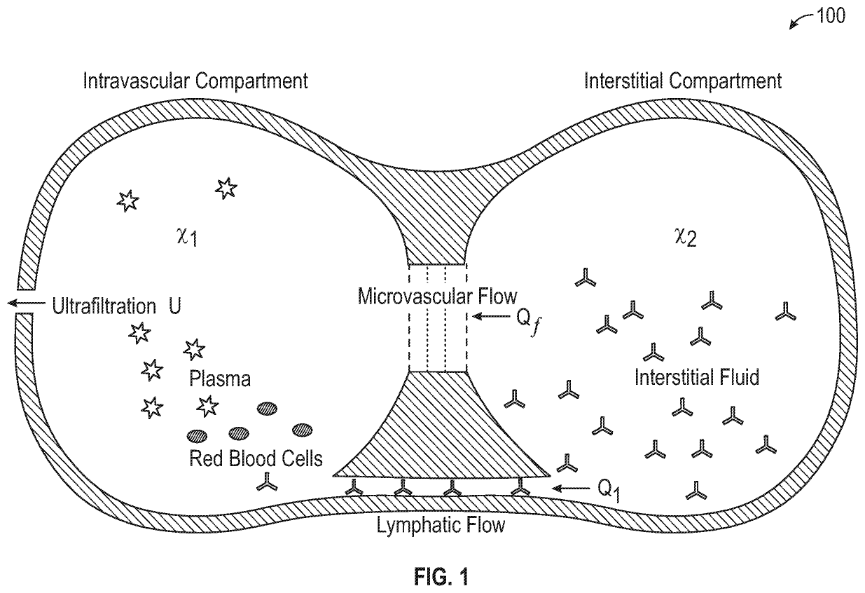

FIG. 1 illustrates a schematic diagram of a two-compartment fluid model describing fluid exchange between interstitial and intravascular compartments separated by a capillary membrane in accordance with some embodiments. The extracellular fluid is modeled by two compartments, intravascular (plasma) and interstitial pools separated by a capillary membrane wall. In an example, only the extracellular fluid dynamics during HD are modeled, and negligible fluid exchange with the intracellular compartment may be ignored. In an example, the model may include the lymphatic system, which returns a small amount of fluid from the interstitial compartment into the intravascular compartment. The model represents ultrafiltration as fluid removed from the intravascular compartment during hemodialysis treatments.

The fluid dynamics of this two-compartment model may be described by the nonlinear differential equations:

$${\dot{x}}_{1} = R ( x ) - u \\ {\dot{x}}_{2} = - R ( x ) \\ H ct = \frac{{V}_{rbc}}{{V}_{rbc} + {x}_{1}} \qquad Eq . 1$$

Where states x₁ and x₂ represent the volumes of the intravascular and interstitial compartments respectively, input u is the ultrafiltration rate, and output Hct is the measured hematocrit which increases proportionally with red blood cell volume V_{rbc} and varies inversely to x₁. The rate R(x) represents the net flow between compartments comprising lymphatic and microvascular flows. This rate is a nonlinear function of the fluid state x and may depend on five parameters V_{rbc}, K_{f}, f, d, and mₚ as described below.

The flow rate between the intravascular and interstitial compartments may be expressed as R=Q₁+Q_{f} where Q_{f} and Q₁ are the microvascular and lymphatic flows respectively. The dependence of Q₁ and Q_{f} on the compartment state x may be described according to the equations below.

Q_{f}=K_{f}(Δₚ−Δ_{π}) Eq. 2

where K_{f} denotes the microvascular refilling/filtration coefficient and p and π represent the hydrostatic and osmotic pressure gradients respectively. In turn, these gradients are described by:

Δₚ=(P_{c}−Pᵢ)

Δ_{π}=(πₚ−πᵢ) Eq. 3

where P_{c}, Pᵢ, p and i denote hydrostatic capillary pressure, interstitial pressure, plasma colloid osmotic pressure, and interstitial colloid osmotic pressure respectively. These pressure are related to the volume state x by:

$${P}_{c} = {P}_{v} + {P}_{o} \\ {P}_{v} = {d ( 100 \frac{{V}_{rbc} + {x}_{1}}{{V}_{rbc} + {V}_{p , eu}} + r )}^{f} \\ {P}_{i} = \{ ( \frac{a 100 {x}_{2}}{{V}_{i , eu}} ) + \frac{b}{( \frac{100 {x}_{2}}{{V}_{i , eu}} + c )} \} \\ {π}_{p} = \{ \frac{{k}_{c 1} {m}_{p}}{{x}_{1}} + \frac{{k}_{c 2} {m}_{p}^{2}}{{x}_{1}^{2}} + \frac{{k}_{c 3} {m}_{p}^{3}}{{x}_{1}^{3}} \} \\ {π}_{i} = \{ \frac{{k}_{c 1} {m}_{i}}{{x}_{2}} + \frac{{k}_{c 2} {m}_{i}^{2}}{{x}_{2}^{2}} + \frac{{k}_{c 3} {m}_{i}^{3}}{{x}_{2}^{3}} \} \qquad Eq . 4$$

where a, c, d, e, f and g are constants and where Pᵥ (Pₒ) denotes venous (offset) pressure, V_{p,eu} (V_{i,eu}) denotes plasma (interstitial) volume for an individual in a euhydration state (e.g., normal fluid volume), and mₚ (mᵢ) denotes protein mass in the plasma (interstitial) volume. The lymphatic flow Q₁ may be defined by

Qₗ(x₂)=g tan h(hPᵢ)+l Eq. 5

where g, h, and l are constants.

These five parameters are identified from individualized clinical data. Identification errors may be accounted for by introducing uncertainty ranges about these estimated parameter values as shown in Table 1.

TABLE 1
TABLE 1 INDIVIDUALIZED MODEL PARAMETERS AND
UNCERTAINTY RANGES

| Parameter | Description | Nominal | Uncertainty range |
|---|---|---|---|
| K_{f} (L/min * mmHg) | filtration coefficient | 0.0057 | [0.0054, 0.006] |
| V_{rbc} (L) | RBC volume | 2 | [1.9, 2.1] |
| D | unitless parameter | 0.01 | [0.0950, 0.1050] |
| F | unitless parameter | 1.46 | [1.387, 1.533] |
| mₚ (g) | plasma protein mass | 210 | [199.5, 220.5] |

In an example, a temporal UFR profile u may be generated according to one or more of the following objectives:

1) remove a given amount of fluid in a given amount of time;

2) satisfy a given, time-dependent, upper-bound on hematocrit (i.e., critical hematocrit profile); (e.g., to provide an implicit lower bound to plasma volume to avoid hypotension)

3) minimize ∥u∥_{∞}; (e.g., to avoid the adverse effects associated with removing too much fluid too quickly)

4) guarantee the above objectives over the parameter ranges in Table 1 (e.g., to guarantee performance when parameter estimation errors occur)

The nonlinear compartment model shown in Eq. 1 taken over the range of parameters in Table 1 includes a family of models. A single UFR profile may be generated that meets all of the above performance specifications when applied to a model in this family (e.g., including each model). For the tractability of this design process respective nonlinear models may be linearized or discretized to produce a corresponding family of linear, discrete-time systems. The associated sampling period is T minutes over which the UFR profile is piecewise constant and has duration of nT minutes. This family of linear systems is described by the difference equation:

$${Hct}_{k} = {Hct}_{0} + {θ}_{3} {\sum }_{i = 0}^{k - 1} {u}_{i} + {θ}_{2} {\sum }_{i = 0}^{k - 1} {θ}_{1}^{( k - 1 - i )} {u}_{i} \qquad Eq . 6$$

where k=1, . . . n, Hct₀ is the initial hematocrit, uₖ denotes u(kT), and where the parameters θ₁, θ₂, and θ₃, accounting for the individualized model parameter uncertainty described in Table I, may take on values in an uncertainty range (e.g., [0.935,0.956], [0.046,0.051], and [0.004,0.008], respectively). In an example, the UFR design may be the solution to the following linear program:

$${minimize}_{{u}_{k} , \overline{u}} \overline{u} \\ \begin{matrix}subject to & 0 \leq {u}_{k} \leq \overline{u} \\ & 0 < \overline{u} \leq {u}_{max} \\ & 0.95 {V}_{T} \leq T {\sum }_{k = 0}^{n - 1} {u}_{k} \leq {V}_{T} \\ & {max}_{θ ∈ Θ} {Hct}_{k} \leq {Hct}_{c , k}\end{matrix} \qquad Eq . 7$$

The first constraint limits ultrafiltration rates to be less than some minimal value which itself has a given physiological bound uₘₐₓ imposed by the second constraint. The third constraint may guarantee at least 95% of target volume of plasma V_{T} will be removed (a minimal relaxation to avoid program infeasibility) while the last constraint may guarantee that a given critical hematocrit profile Hct_{c,k} is not exceeded over all model parameters θ. The maximization of Hctₖ in the last constraint may be achieved for all k when the components of θ take on their largest values.

The UFR profile uₖ described above may be based on a simplified model, (e.g., a linearized version of Eq. 1, sampled at time intervals of length T). Applying a zero-order hold (ZOH) to uₖ the continuous-time profile u(t), t>0 may be obtained, which is used in the dialysis setting. The performance of uₖ may be guaranteed for the reduced model, whereas u(t) may be applied to the full nonlinear, continuous-time model of Eq. 1. In an example, two requisite clinical conditions are be satisfied: (i) the target amount of fluid (within 5%) is withdrawn during the given dialysis period, and (ii) the hematocrit is kept below a (time-invariant) specified critical value.

In an example, uₖ is a monotone non-increasing sequence, uₖ>uₖ+1, and u(t) is thus likewise monotone non-increasing. In an example, discussion related to the systems and methods described herein may be related to arbitrary nonnegative, monotone non-increasing uₖ and u(t).

In an example, for any UFR profile u(t), an amount of fluid removed during a time interval [0,t] may be defined according to the equation below:

U(t)≡∫₀^{t}u(τ)dr Eq. 8

When u(t)derived from a sequence uk by ZOH, then, I(nT)=Σ₀ⁿ⁻¹uₖ, and thus 0.95V_{T}≤U(Nt)≤V_{T}, where the third condition described above is satisfied, and assertion (i) is satisfied.

Let E₀ be the equilibrium set of Eq. 1 when the UFR is “turned off”, (e.g., u(t)≡0, E₀={x : R(x)=0}. Due to the nonlinearity of R(x), E₀ is evaluated numerically. E₀ is the graph of a monotone increasing continuous function that divides the open first quadrant into two regions where R>0 and R<0. Next, a rectangle B=[x_{1L}, x_{1H}]×[x_{2L}, x_{2H}] is chosen in the open first quadrant with lower left corner (x_{1L}, x_{2L}) and upper right come (x_{1H}, x_{2H}) both in E₀. The function R(x) is continuous in B and the partial derivatives satisfy:

$${R}_{1} = \frac{\partial R}{\partial {x}_{1}} < 0 \\ {R}_{2} = \frac{\partial R}{\partial {x}_{2}} > 0 \qquad Eq . 9$$

Let E+ denote the vascular refilling region, E+≡{xϵB : R(x)>0}.

In an example, x(t) is a solution of Eq. 1 with initial condition x₀=(x₁₀, x₂₀), and with u(t)≥0, 0≤t≤t_{f} an arbitrary monotone non-increasing UFR profile, where, for example, t_{f}>0 the duration of the dialysis session, in an example, V(t)=x₁(t)+x₂(t), and V(t) is the total fluid volume in the system at time t. In an example, r(t)≡R(x(t)), where r(t) is the flow rate into the vascular compartment at time t, and rᵢ(t)=Rᵢ(x(t)), I=1, 2. Results of the full nonlinear system under the designed. UFR profile are described below. In an example, the results do not use any properties of R(x) beyond those in the previous paragraph and they may hold for a large class of UFR profiles. In theorem 1 (shown below), and under appropriate initial conditions, solutions x(t) of the full model in :Eq. 1 remain within a physiologically reasonable region.

In an example, r(t₀)=v>0 for some t₀ϵ[0, t_{f}] and u(t)≤v for t≥t₀. In this example, r(t)≤v for t≥t₀. This result may not require monotoni city of ⁻u(t). Theorem 1 states, Let a_{2L} be the solution of R(x_{1L}, a_{2L})=u(0) and assume U(t_{f})<V(0)−(x_{1L}+a_{2L}). Let x(t) be a solution of Eq. 1 with x₀ϵB. When 0≤R(x₀)≤u(0), then, for 0<t≤t_{f}, x(t) stays in the region Cᵣ⊂B bounded by Eᵤ₍₀₎, E₀, and the line L_{tf}, having slope-1 and x1-intercept V(0)−U(t_{f}). When R(x₀)>u(0), then x(t) ϵB for 0<t≤t_{f}, the same conclusion holds if R(x₀)<0.

In Theorem 2 below, a first part 2a describes a global condition, under which a UFR profile u(t) keeps Hct under a specified time-invariant critical value. In part 2a, bᵢ corresponds to the plasma volume corresponding to the critical Hct value. In part 2b a stepwise condition is described that is easier to satisfy and under which the conclusion holds. In part 2c a restatement of part 2b is described. Additionally, a Lemma 2 to Theorem 2 is provided.
Let 0>t1≤tf. Given b₁ in the interval [x_{1L}, x_{1H}], let b₂ be the solution of R(b₁, b₂)=u(t0). When x₁(t₀)>b₁ and U(t₁)<V(0)−(b₁+b₂), then x₁(t)>b₁ for t₀<t≤t₁. Lemma 2b: Let (c₁, c₂) solve the equations R(c₁, c₂)=u(t0) and c₁+c₂=V(0) −U(t₁). For any b₁<c₁, when x₁(t₀)>b₁, then x₁(t)>b₁ for t₀<t≤t₁.

### Lemma 2

Let (b₁, b₂) be as in Lemma 2 with t0=0. When x₁(0)>b₁ and U(t_{f})<V(0)−(b₁+b₂), then x₁(t)>b₁ for 0<t≤t_{f}.

### Theorem 2, Part 2a

Let Tₖ=Kt, k=0, . . . , n, and, for each k, let b₂ₖ solve the equation R(b₁, b₂ₖ)=uₖ, and let Vₖ=b₁+b₂ₖ. In an example, u(t) is obtained from the sequence uₖ by applying ZOH over the intervals [T₅, Tₖ₊₁]. When U(Tₖ₊₁)<V(0)−Vₖ for each k and x₁(0)>b₁, then x₁(t)>b₁ for 0<t≤t_{f}.

### Theorem 2, Part 2b

For each k, let (c₁ₖ, c₂ₖ) solve the equations R(c₁ₖ, c₂ₖ)=uₖ, and c₁ₖ+c₂ₖ=V(0) −U(Tₖ₊₁). When b₁<c₁ₖ for every k, and x₁(0)>b₁, then x₁(t)>b₁ for 0<t≤t_{f}.

### Theorem 2, Part 2c

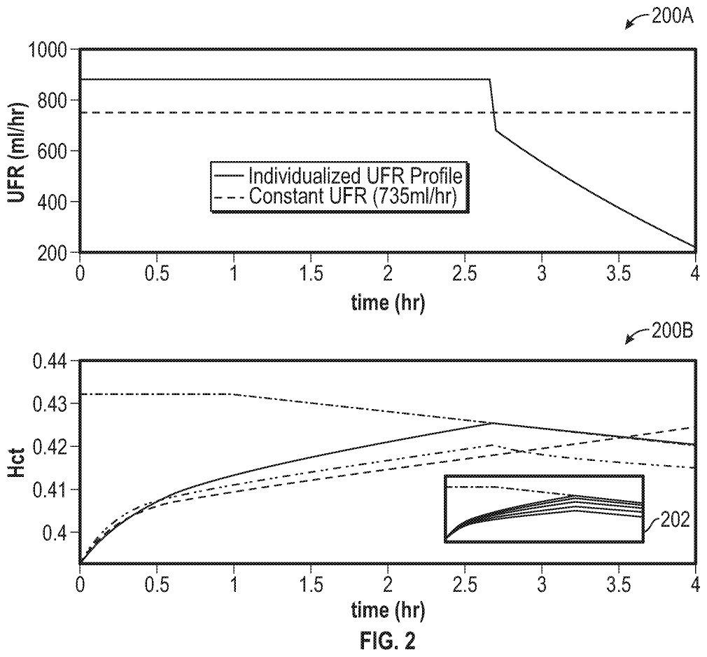

FIG. 2 illustrates an example ultrafiltration rate profile and hematocrit information over time in accordance with some embodiments.

The graph 200A illustrates a designed UFR. profile (blue solid) and a constant UFR profile (blue dashed). The graph 200B illustrates the prescribed Hct constraint (red solid), the worst-case linear Hct response (blue solid), the worst-case nonlinear Hct response (green solid), and the worst-case nonlinear Hct response with constant UFR profile (blue dashed).

TABLE 2
MODEL PARAMETERS

| Parameter | Value | Parameter | Value |
|---|---|---|---|
| a (unitless) | 0.006 | K_{f}L/(min * mmHg) | 0.0057 |
| b (unitless) | −198 | mₚ (g) | 210 |
| c (unitless) | −45 | mᵢ (g) | 210 |
| d (unitless) | 0.01 | Pₒ (mmHg) | 13.128 |
| r (unitless) | −30 | V_{i,eu} (L) | 11 |
| f (unitless) | 1.46 | V_{p,eu} (L) | 3 |
| g (unitless) | 0.045 | k_{c1} (unitless) | 0.21 |
| h (unitless) | 0.7672 | k_{c2} (unitless) | 0.0016 |
| l (unitless) | 0.045 | k_{c3} (unitless) | 9e−6 |
| V_{rbc} (L) | 2 | — | — |

To illustrate the UFR design, the model described by Eq. 1 may be used with parameters described in Tables 1 and 2. For the constrained optimization program described in Eq. 7, an example may use uₘₐₓ=975 ml/hr, V_{T}=3 liters, n=240 (e.g., t_{f}=4 hrs), the maximizing θ=(0965, 0.051, 0.008, respectively as discussed above), and Hct_{c} as shown in graph 200B (red solid).

The specific form of this hematocrit profile Hct_{c} is described in more detail below. In an example, a UFR bound or guideline may include limiting an ultrafiltration rate not to exceed 1975 ml/hr to minimize cardiovascular risk. The UFR profile shown in FIG. 2 (graph 200A, with the blue-solid line) removes an acceptable 2.94 L of fluid. An example worst-case Hct response to this UFR for both the linearized (graph 200B, blue-solid line) and nonlinear systems (graph 200B, green-solid line) satisfy the critical Hct constraint (graph 200B, red-solid line). This is expected in the linear case (by design), but may not be guaranteed for the nonlinear system. In contrast, the constant UFR profile of 0.735 liters/hr (graph 200A, blue-dashed line), removes the same 2.94 L over 4 hours, but has a worst-case nonlinear response that violates the hematocrit constraint (graph 200B, blue-dashed line). Finally, in the bottom inset 202 of FIG. 2, a response of the linear model Eq. 6 is simulated for all admissible parameters in Tables 1 and 2, illustrating the robustness of the 1J⁻FR design.

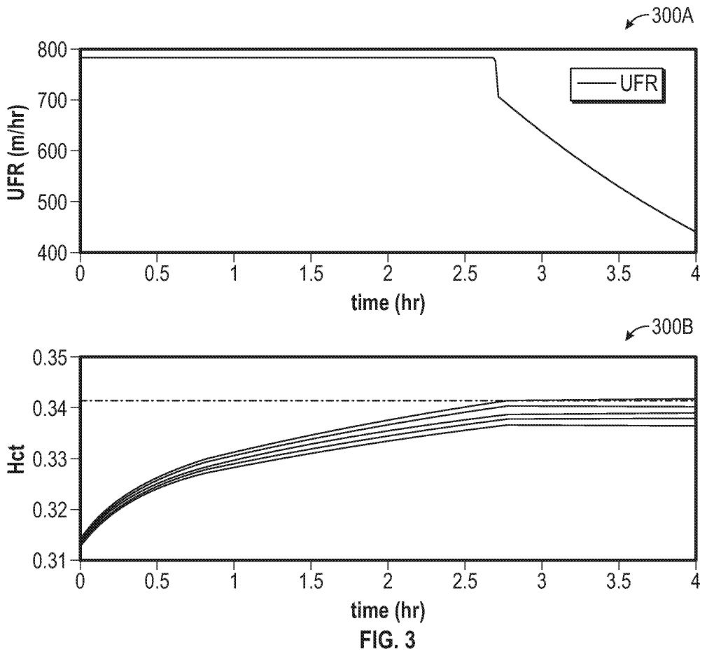

FIG. 3 illustrates robustness hematocrit information for an ultrafiltration rate profile over time in accordance with some embodiments.

A UFR profile is illustrated in graph 300A (green line), Robust discrete-time Hct responses (blue lines) and critical Hct (dashed, red line) are shown in graph 300B. In this example, the regression model was run using 30 data points, to estimate the parameters. To account for the high variability of the physiological parameters and the possible estimation error, a ±10% of change for the estimated parameters V_{rbc}, K_{f}, x₁₀, and X₂₀ may be used to create uncertainty ranges for these parameters. In the design of the robust, optimal ultrafiltration rate profile, the critical hematocrit level, y_{c}, may be chosen as an increase in the initial value by (109%), (e.g., y_{c}=y₀+0.9* y₀). The optimal ultrafiltration rate profile may be calculated by solving the optimization problem.

FIG. 3 shows the robustness performance of the UFR profile design where the hematocrit response to the optimal ultrafiltration rate profile cannot exceed the critical value, y_{c}, for any given parameter set (e.g., θ₁, θ₂, and θ₃, in uncertainty ranges (e.g., [0.935,0.956], [0.046,0.051], and [0.004,0.008], respectively). As seen, the blue lines representing the Hct responses are below or at the red line representing the critical Hct.

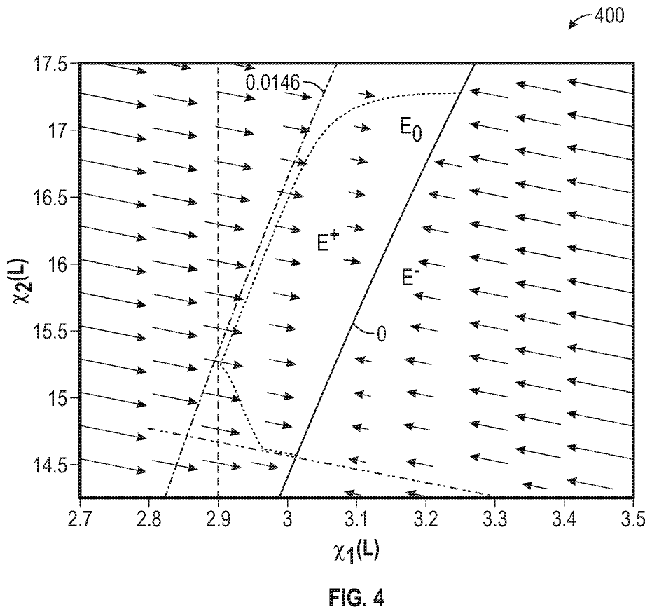

FIG. 4 illustrates boundary information for a state trajectory of a nonlinear model of an ultrafiltration rate in accordance with some embodiments.

In FIG. 4, x₁*=2.9 L (shown by green dashed-line) is the maximum critical plasma volume, which corresponds to yᵥ(n)=107% * y₀, where y_{c}(n) is the lowest Hct level within the critical Hct profile, n=240 min, and V_{rbc}=2.1 L (e.g., maximum value). In this case, Theorem 1 is satisfied for all k, indicating that the hematocrit of the nonlinear model in Eq. 1 cannot violate the critical hematocrit profile as shown in FIG. 2 (graph 200B, blue-solid line). In an example, conditions guaranteeing that the nonlinear Hct satisfies critical Hct profile may be checked before simulation.

In an example, when y_{c}(n)=106% * y₀, then the corresponding maximum critical plasma volume is x₁*=2.95 L. Using this value and the new UFR profile, the local conditions in Theorem 1 are violated fork between 100-180 min, but in this period of time the critical Hct is not violated, which can be verified by checking these conditions for each x₁* corresponding to each k between 100-180 min. Therefore, by decreasing x from 2.95 L to 2.89 L, which corresponds to 107.3% * y₀, the conditions in Theorem 1 are satisfied for all k.

As stated, while the worst-case nonlinear Hct response in FIG. 2 (graph 200B, green) does satisfy the time-varying Hct constraint, this is not guaranteed for the linearized system. In Theorems 1 and 2, guarantees for the nonlinear system are provided, and are illustrated below. First, the nonlinear Hct trajectory remains within the physiologically reasonable region B=[2, 5] [11, 28.6]. The initial volume state is determined at equilibrium such that R(x₀)=0. Thus, Theorem 1 is in force, and for 0<t≤t_{f}, x(t) stays in the region Cᵣ⊂B bounded by Eᵤ₍₀₎, E₀, and the line L_{tf} having slope-1 and x1-intercept V(0)−U(t_{f}). Cᵣ is the region described in FIG. 4 by the solid-blue line (E₀), black-dashed line (R(u)) and green-dashed (L_{tf}) line.

The robustness of UFR profiles described above may apply to the system obtained by linearization of model Eq. 1 about an equilibrium point and discretization of the time. The continuous-time profile u(t) is obtained by applying a zero-order hold to the discrete profile uₖ, therefore the target volume removed will be the same for u(t) as for uₖ. Because Theorems 1 and 2 use broad qualitative properties of R, they apply to the continuous-time linearized system, which therefore also respects the Hct constraint.

Further, for linearization and discretization of Eq. 1:

### A. Linearization

For u≡0, let xₛₛ, be the equilibrium state of the nonlinear compartmental model (1). With δx=x−xₛₛ; δu=u; and δH ct=H ct=H ct₀, we form the linearized system (around xₛₛ and u≡0)

$$\begin{matrix} δ \dot{x} = A δ x + B δ u ; \\ δ Hct = C δ x \\ and where & Eq . 10 \\ A = [ \begin{matrix}- α & β \\ α & - β\end{matrix} ] ; B = [ \begin{matrix}- 1 \\ 0\end{matrix} ] ; C = [ \begin{matrix}- K & 0 ] .\end{matrix} \\ where & Eq . 11 \\ K = \frac{{V}_{rbc}}{{( {V}_{rbc} + {x}_{1 ss} )}^{2}} ; \\ α = \frac{{K}_{f} df 100}{{V}_{rbc} + {V}_{p , eu}} {( 100 \frac{{V}_{rbc} + {x}_{1 ss}}{{V}_{rbc} + {V}_{p , eu}} )}^{f - 1} + {K}_{f} \{ \frac{{k}_{c 1} {m}_{p}}{{x}_{1 ss}^{2}} + \frac{2 {k}_{c 2} {m}_{p}^{2}}{{x}_{1 ss}^{3}} + \frac{3 {k}_{c 3} {m}_{p}^{3}}{{x}_{1 ss}^{4}} \} ; \\ β = {K}_{f} \{ \frac{{k}_{c 1} {m}_{i}}{{x}_{2 ss}^{2}} + \frac{2 {k}_{c 2} {m}_{i}^{2}}{{x}_{2 ss}^{3}} + \frac{3 {k}_{c 3} {m}_{i}^{3}}{{x}_{2 ss}^{4}} + \frac{100}{{V}_{i , eu}} ( a - \frac{b}{( 100 \frac{{x}_{2 ss}}{{V}_{i , eu}} + c )} ) \} + g {sech}^{2} ( ha 100 \frac{{x}_{2 ss}}{{V}_{i , eu}} + \frac{hb}{( 100 \frac{{x}_{2 ss}}{{V}_{i , eu}} + c )} ) & Eq . 12\end{matrix}$$

The transfer function G(s)=δH ct(s)/δu(s) is then

$$G ( s ) = \frac{K ( s + β )}{s ( s + α + β )} \qquad Eq . 13$$

B. Discretization: Consider the sequence δHctₖ obtained by sampling the output of Eq. 10, e.g., δHctₖ=δHct (kT) for given sampling period T. Let δu be piecewise constant over these sampling periods as in δu(t)=vₖ when kT<t≤(k+1)T for e given sequence vₖ. The linearized volume state δxₖ, then evolves according to the discrete-time equations:

δxₖ₊₁=Φδxₖ+Γvₖ;

δH ctₖ=Cδxₖ Eq. 14

where

Φ=e^{AT}; Γ=∫₀^{T}e^{As}dsB. Eq. 15

The solution to Eq. 14 can then be written in typical fashion as

$$δ {Hct}_{k} = {Φ}^{k} δ {x}_{0} + {\sum }_{i = 0}^{n - 1} {h}_{i}^{n - 1 - i} v ( i ) \qquad Eq . 16$$

where the impulse response hk is the inverse z-transform of C(zI-Φ)⁻¹Γ. Computation gives (2) where

$${θ}_{1} = {e}^{- ( α + β ) T} ; \\ {θ}_{2} = \frac{K α}{{( α + β )}^{2}} ( 1 - {e}^{- ( α + β ) T} ) ; \\ {θ}_{3} = T \frac{K β}{( α + β )} \qquad Eq . 17$$

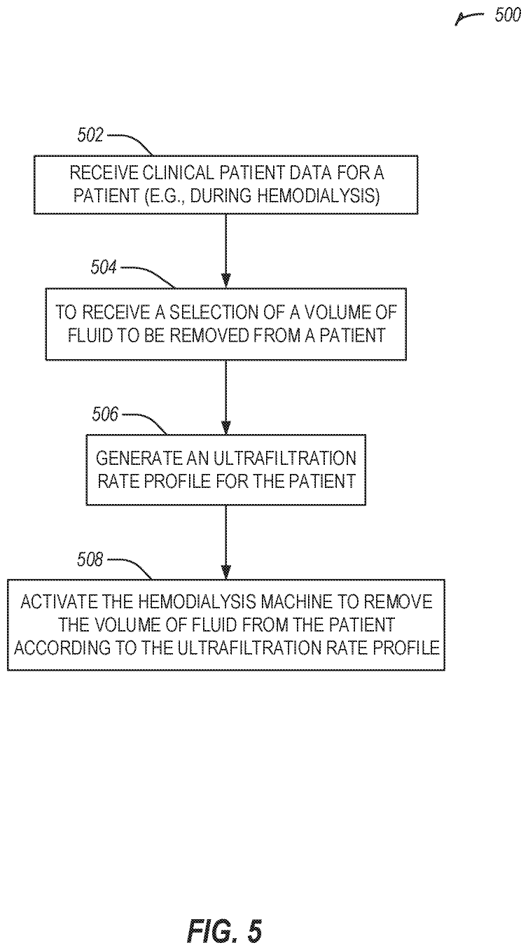

FIG. 5 illustrates a flow chart showing a technique 500 for removing fluid from a patient at a hemodialysis machine in accordance with some embodiments. The technique 500 may be stored using at least one non-transitory machine-readable medium. The machine-readable medium may include instructions, which when executed by at least one processor or a machine, cause the at least one processor or the machine to perform the technique 500.

The technique 500 includes an operation 502 to receive clinical patient data (e.g., during hemodialysis), for example for a patient at a previous session (e.g., generated before a current session). The technique 500 includes an operation 504 to receive a selection (e.g., from a doctor) of a volume of fluid to be remove from the patient, such as in the current session. The selection may include a specified period of time, or the specified period of time may be determined from a default period of time.

The technique 500 includes an operation 506 to generate an ultrafiltration rate profile for the patient, such as based on the clinical patient data, the volume of fluid to be removed, or a patient-specific fluid volume model, for example within the period of time. In an example, the ultrafiltration rate profile may be time-variant (e.g., change the rate over the specified period of time or the current session). The ultrafiltration rate profile may limit hematocrit in the patient. For example, the hematocrit may be kept under a threshold throughout the current session or during the period of time while the hemodialysis machine removes the volume of fluid. In an example, the ultrafiltration rate profile minimizes, over the period of time, a maximal ultrafiltration rate for the patient while removing the volume of fluid over the period of time.

Operation 506 may include using a nonlinear fluid-volume model as the patient-specific fluid volume model, including an intravascular pool and an interstitial pool. For example, states of the nonlinear fluid-volume model may be calculated to be within a specified physiological region. In an example, states of the nonlinear fluid-volume model are subject to a time-varying hematocrit constraint. Operation 506 may include generating the ultrafiltration rate profile using a linearized version of the nonlinear fluid-volume model based on a zero-order hold over a sampling period.

The technique 500 includes an operation 508 to activate the hemodialysis machine to remove the volume of fluid from the patient, such as during the period of time, according to the ultrafiltration rate profile.

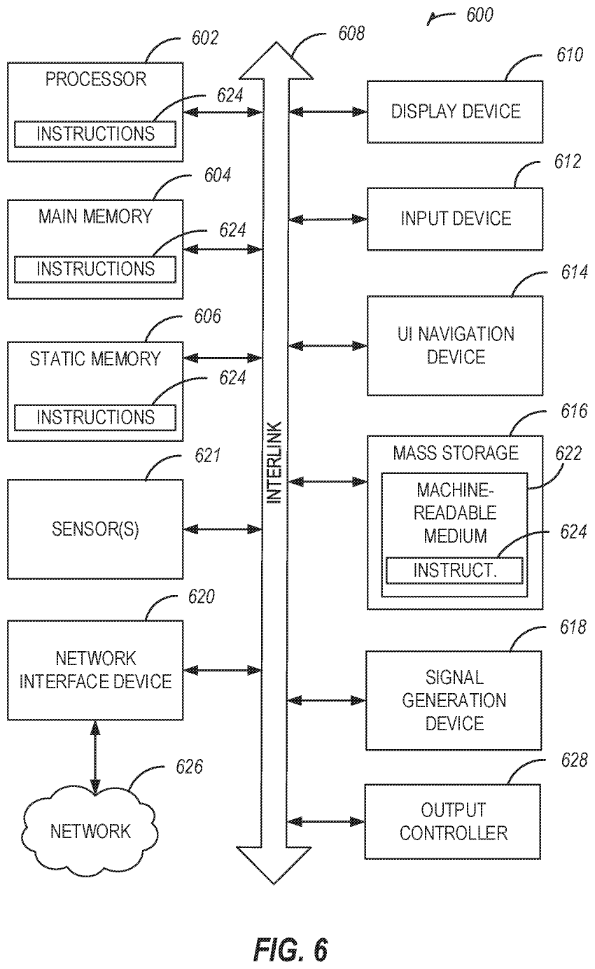

FIG. 6 illustrates a block diagram of an example machine 600 upon which any one or more of the techniques discussed herein may perform in accordance with some embodiments. In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to he taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NIT), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium ©r multiple media (e.g., a centralized or distributed database, and/or associated caches and servers) configured to store the one or more instructions 624. The term “machine readable medium” may include any medium that is capable of storing, encoding, or carrying instructions fur execution by the machine 600 and that cause the machine 600 to perform any one or more of the techniques of the present disclosure, or that is capable of storing, encoding or carrying data structures used by or associated with such instructions. Non-limiting machine readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MEM), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Each of these non-limiting examples may stand on its own, or may be combined in various permutations or combinations with one or more of the other examples.

Example 1 is a hemodialysis machine for removing fluid from a patient, the hemodialysis machine comprising: memory including instructions, which when executed by a processor of the hemodialysis machine, cause the processor to: receive clinical patient data generated before a current session; receive a user selection of a volume of fluid to be removed in the current session within a specified period of time; generate a time-variant ultrafiltration rate profile for the patient based on the clinical patient data, the volume of fluid to be removed within the specified period of time, and a patient-specific fluid volume model; and activate the hemodialysis machine to remove the volume of fluid from the patient during the specified period of time according to the ultrafiltration rate profile.

In Example 2, the subject matter of Example 1 includes, wherein the ultrafiltration rate profile limits hematocrit in the patient to under a threshold throughout the current session while the hemodialysis machine removes the volume of fluid.

In Example 3; the subject matter of Examples 1-2 includes, wherein the ultrafiltration rate profile minimizes, over the period of time, a maximal ultrafiltration rate for the patient while removing the volume of fluid over the period of time.

In Example 4, the subject matter of Examples 1-3 includes, wherein the patient-specific fluid volume model is a nonlinear fluid-volume model including an intravascular pool and an interstitial pool.

In Example 5, the subject matter of Example 4 includes, wherein states of the nonlinear fluid-volume model are calculated to be within a specified physiological region.

In Example 6, the subject matter of Examples 4-5 includes, wherein states of the nonlinear fluid-volume model are subject to a time-varying hematocrit constraint.

In Example 7, the subject matter of Examples 4-6 includes, wherein to generate the ultrafiltration rate profile, the processor is further to use a linearized version of the nonlinear fluid-volume model based on a zero-order hold over a sampling period.

Example 8 is a non-transitory machine-readable medium including instructions for removing fluid from a patient, which when executed by a processor of a hemodialysis machine, cause the processor to: receive clinical patient data generated before a current session; receive a user selection of a volume of fluid to be removed in the current session within a specified period of time; generate a time-variant ultrafiltration rate profile for the patient based on the clinical patient data, the volume of fluid to be removed within the specified period of time, and a patient-specific volume model; and activate the hemodialysis machine to remove the volume of fluid from the patient during the specified period of time according to the ultrafiltration rate profile.

In Example 9, the subject matter of Example 8 includes, wherein the ultrafiltration rate profile limits hematocrit in the patient to under a threshold throughout the current session while the hemodialysis machine removes the volume of fluid.

In Example 10, the subject matter of Examples 8-9 includes, wherein the ultrafiltration rate profile minimizes, over the period of time, a maximal ultrafiltration rate for the patient while removing the volume of fluid over the period of time.

In Example 11, the subject matter of Examples 8-10 includes, wherein the patient-specific fluid volume model is a nonlinear fluid-volume model including an intravascular pool and an interstitial pool.

In Example 12, the subject matter of Example 11 includes, wherein states of the nonlinear fluid-volume model are calculated to be within a specified physiological region.

In Example 13, the subject matter of Examples 11-12 includes, wherein states of the nonlinear fluid-volume model are subject to a time-varying hematocrit constraint.

In Example 14, the subject matter of Examples 11-13 includes, wherein to generate the ultrafiltration rate profile, the instructions further cause the processor to use a linearized version of the nonlinear fluid-volume model based on a zero-order hold over a sampling period.

Example 15 is a method for removing fluid from a patient at a hemodialysis machine, the method comprising: receiving clinical patient data generated before a current session; receiving a user selection of a volume of fluid to be removed in the current session within a specified period of time; generating a time-variant ultrafiltration rate profile for the patient based on the clinical patient data, the volume of fluid to be removed within the specified period of time, and a patient-specific fluid volume model; and activating the hemodialysis machine to remove the volume of fluid from the patient during the specified period of time according to the ultrafiltration rate profile.

In Example 16, the subject matter of Example 15 includes, wherein the ultrafiltration rate profile limits hematocrit in the patient to under a threshold throughout the current session while the hemodialysis machine removes the volume of fluid.

In Example 17, the subject matter of Examples 15-16 includes, wherein the ultrafiltration rate profile minimizes, over the period of time, a maximal ultrafiltration rate for the patient while removing the volume of fluid over the period of time.

In Example 18, the subject matter of Examples 15-17 includes, wherein the patient-specific fluid volume model is a nonlinear fluid-volume model including an intravascular pool and an interstitial pool.

In Example 19, the subject matter of Example 18 includes, wherein states of the nonlinear fluid-volume model are calculated to be within a specified physiological region.

In Example 20, the subject matter of Examples 18-19 includes, wherein states of the nonlinear fluid-volume model are subject to a time-varying hematocrit constraint.

In Example 21, the subject matter of Examples 18-20 includes, wherein generating the ultrafiltration rate profile includes using a linearized version of the nonlinear fluid-volume model based on a zero-order hold over a sampling period.

Example 22 is at least one machine-readable medium including instructions that, when executed by processing circuitry, cause the processing circuitry to perform operations to implement of any of Examples 1-21.

Example 23 is an apparatus comprising means to implement of any of Examples 1-21.

Example 24 is a system to implement of any of Examples 1-21.

Example 25 is a method to implement of any of Examples 1-21.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A hemodialysis machine for removing fluid from a patient, the hemodialysis machine comprising:
memory including instructions, which when executed by a processor of the hemodialysis machine, cause the processor to:
receive clinical patient data generated before a current session;
receive a user selection of a volume of fluid to be removed in the current session within a specified period of time;
generate a time-variant ultrafiltration rate profile for the patient based on the clinical patient data, the volume of fluid to be removed within the specified period of time, and a patient-specific fluid volume model; and
activate the hemodialysis machine to remove the volume of fluid from the patient during the specified period of time according to the ultrafiltration rate profile.

2. The hemodialysis machine of claim 1, wherein the ultrafiltration rate profile limits hematocrit in the patient to under a threshold throughout the current session while the hemodialysis machine removes the volume of fluid.

3. The hemodialysis machine of claim 1, wherein the ultrafiltration rate profile minimizes, over the period of time, a maximal ultrafiltration rate for the patient while removing the volume of fluid over the period of time.

4. The hemodialysis machine of claim 1, wherein the patient-specific fluid volume model is a nonlinear fluid-volume model including an intravascular pool and an interstitial pool.

5. The hemodialysis machine of claim 4, wherein states of the nonlinear fluid-volume model are calculated to be within a specified physiological region.

6. The hemodialysis machine of claim 4, wherein states of the nonlinear fluid-volume model are subject to a time-varying hematocrit constraint.

7. The hemodialysis machine of claim 4, wherein to generate the ultrafiltration rate profile, the processor is further to use a linearized version of the nonlinear fluid-volume model based on a zero-order hold over a sampling period.

8. A non-transitory machine-readable medium including instructions for removing fluid from a patient, which when executed by a processor of a hemodialysis machine, cause the processor to:
receive clinical patient data generated before a current session;
receive a user selection of a volume of fluid to be removed in the current session within a specified period of time;
generate a time-variant ultrafiltration rate profile for the patient based on the clinical patient data, the volume of fluid to be removed within the specified period of time, and a patient-specific volume model; and
activate the hemodialysis machine to remove the volume of fluid from the patient during the specified period of time according to the ultrafiltration rate profile.

9. The machine-readable medium of claim 8, wherein the ultrafiltration rate profile limits hematocrit in the patient to under a threshold throughout the current session while the hemodialysis machine removes the volume of fluid.

10. The machine-readable medium of claim 8, wherein the ultrafiltration rate profile minimizes, over the period of time, a maximal ultrafiltration rate for the patient while removing the volume of fluid over the period of time.

11. The machine-readable medium of claim 8, wherein the patient-specific fluid volume model is a nonlinear fluid-volume model including an intravascular pool and an interstitial pool.

12. The machine-readable medium of claim 11, wherein states of the nonlinear fluid-volume model are calculated to be within a specified physiological region.

13. The machine-readable medium of claim 11, wherein states of the nonlinear fluid-volume model are subject to a time-varying hematocrit constraint.

14. The machine-readable medium of claim 11, wherein to generate the ultrafiltration rate profile, the instructions further cause the processor to use a linearized version of the nonlinear fluid-volume model based on a zero-order hold over a sampling period.

15. A method for removing fluid from a patient at a hemodialysis machine, the method comprising:
receiving clinical patient data generated before a current session;
receiving a user selection of a volume of fluid to be removed in the current session within a specified period of time;
generating a time-variant ultrafiltration rate profile for the patient based on the clinical patient data, the volume of fluid to be removed within the specified period of time, and a patient-specific fluid volume model; and
activating the hemodialysis machine to remove the volume of fluid from the patient during the specified period of time according to the ultrafiltration rate profile.

16. The method of claim 15, wherein the ultrafiltration rate profile limits hematocrit in the patient to under a threshold throughout the current session while the hemodialysis machine removes the volume of fluid.

17. The method of claim 15, wherein the ultrafiltration rate profile minimizes, over the period of time, a maximal ultrafiltration rate for the patient while removing the volume of fluid over the period of time.

18. The method of claim 15, wherein the patient-specific fluid volume model is a nonlinear fluid-volume model including an intravascular pool and an interstitial pool.

19. The method of claim 18, wherein states of the nonlinear fluid-volume model are calculated to be within a specified physiological region.

20. The method of claim 18, wherein states of the nonlinear fluid-volume model are subject to a time-varying hematocrit constraint.
